(19) United States
(12) Patent Application Publication
(10) Pub. No.: US 2016/0310418 A1
(43) Pub. Date: Oct. 27, 2016

(54) CRUSH-RESISTANT SOLID ORAL DOSAGE FORM

(71) Applicant: SUN PHARMACEUTICAL INDUSTRIES LIMITED, Mumbai, Maharashtra (IN)

(72) Inventors: Anuj Kumar FANDA, Ghaziabad (IN); Kumaravel VIVEK, Chennai (IN); Ravesh Kumar SHARMA, Khargone (IN); Vinay Kumar GARG, Gurgaon (IN); Romi Barat SINGH, Varanasi (IN)

(73) Assignee: SUN PHARMACEUTICAL INDUSTRIES LIMITED, Mumbai, Maharashtra (IN)

(21) Appl. No.: 15/103,570

(22) PCT Filed: Dec. 9, 2014
(86) PCT No.: PCT/IB2014/066736

§ 371 Date: Jun. 10, 2016

(30) Foreign Application Priority Data
Dec. 11, 2013 (IN) 3611/DEL/2013

(51) Int. Cl. A61K 9/68; A61K 31/138; A61K 9/20; A61K 9/28; A61K 47/34; A61K 9/48

(52) U.S. Cl. CPC A61K 9/0058; A61K 47/34; A61K 9/4833; A61K 9/4808; A61K 9/4866; A61K 9/2095; A61K 9/2054; A61K 9/2031; A61K 9/2013; A61K 9/2866; A61K 9/2893; A61K 9/2018; A61K 31/138

(57) ABSTRACT

The present invention relates to a method of imparting crush-resistance to an oral solid dosage form comprising a drug prone to abuse, so as to prevent the unintended and illicit use of the dosage form.

### FIELD OF THE INVENTION

The present invention relates to a method of imparting crush-resistance to an oral solid dosage form comprising a drug prone to abuse, so as to prevent the unintended and illicit use of the dosage form.

### BACKGROUND OF THE INVENTION

Abuse potential refers to the use of a drug in non-medical situations, repeatedly or even sporadically, for its positive psychoactive effects. These drugs are characterized by their central nervous system (CNS) activity. Hence, the most common drugs prone to abuse include opioid analgesics, CNS stimulants, and CNS depressants. These drugs may produce psychic or physical dependence and may lead to addiction, which promotes drug-seeking behavior. Drug abusers and/or addicts typically may use any of the means (e.g., crush, shear, grind, chew, dissolve, heat, extract, and/or otherwise damage the product) to obtain a significant amount or even the entire amount of the drug available by 1) injection, 2) inhalation, and/or 3) oral consumption for immediate absorption.

Several attempts have been made to diminish abuse of orally administered drugs.

U.S. Pat. No. 4,070,494 discloses adding a swellable agent to the dosage form in order to prevent abuse. When water is added to extract the drug, this agent swells and ensures that the filtrate separated from the gel contains only a small quantity of drug.

PCT Publication No. WO 1995/020947 discloses a multilayer tablet based on a similar approach of preventing parenteral abuse, said tablet containing the drug prone to abuse and at least one gel-former, each in different layers.

Another known strategy for abuse deterrent formulations involves adding an antagonist to the drug in the dosage form, e.g., antagonists such as naloxone or naltrexone are added in the case of opioids.

U.S. Pat. No. 7,201,920 discloses compositions comprising an opioid analgesic, a gel-forming polymer, a nasal tissue irritant, and an emetic for deterring abuse of the drug. It uses aversive substances which are otherwise inert, but produce an unacceptable reaction and irritation when tampered with and administered by the unintended route. Specifically disclosed aversive substances are sodium lauryl sulfate used as a nasal tissue irritant and zinc sulfate used as an emetic.

U.S. Publication No. 2010/0249045 discloses a dosage form for oral administration, the dosage form comprising: a) a drug combined with, b) at least two abuse-deterring ingredients selected from the group consisting of: (i) hydrogenated vegetable oils; (ii) polyoxyethylene stearates; (iii) glycerol monostearate; and (iv) poorly water soluble waxes having a melting point in the range from 45° C. to 100° C.

U.S. Pat. No. 8,114,383 discloses an abuse-proof, thermoformed dosage form containing one or more drugs prone to abuse together with physiologically acceptable auxiliary substances, and at least one synthetic or natural polymer with a breaking strength of at least 500 N.

Nonetheless, there is a continuing need for an alternative delivery system which deters abuse and minimizes or reduces the potential for physical or psychological dependence, through all possible routes such as oral, parenteral, and nasal.

### SUMMARY OF THE INVENTION

The present invention relates to a method of imparting crush-resistance to an oral solid dosage form comprising a drug prone to abuse, so as to prevent the unintended and illicit use of the dosage form.

### DETAILED DESCRIPTION OF THE INVENTION

A first aspect of the present invention provides a crush-resistant oral solid dosage form, comprising:

a) a therapeutically effective amount of a drug prone to abuse; and

b) a chewing gum base comprising a chewable plastic polymer;

wherein the amount of said gum base is such that it causes the dosage form to deform plastically without breaking into a powder when said dosage form is tampered for abuse.

According to one embodiment of this aspect, the chewable plastic is selected from the group comprising polyisobutylene, butadiene styrene, polyvinyl acetate, terpene resins, ester gums, ethylene vinyl acetate, and mixtures thereof.

According to one embodiment of this aspect, the chewing gum base is present in an amount from about 20% w/w to about 85% w/w of the composition.

According to one more embodiment of this aspect, the solid dosage form further comprises a release-controlling polymer.

According to another embodiment of this aspect, the release-controlling polymer is selected from the group comprising hydroxypropylmethyl cellulose, hydroxyethyl cellulose, polyethylene glycol, polyethylene oxide, hydroxypropyl cellulose, carboxymethyl cellulose, cellulose ethers, cellulose esters, polymethacrylic acid esters copolymers, aminoalkyl methacrylate copolymers, copolymers of polyvinyl acetate and polyvinyl pyrrolidone, polyvinyl alcohol, glyceryl behenate, carnauba wax, xanthan gum, starch, sodium alginate, tragacanth, poloxamers, carbomers, and mixtures thereof.

According to another embodiment of this aspect, the chewing gum base is present in an amount from about 20% w/w to about 85% w/w, and the release-controlling polymer is present in an amount from about 10% w/w to about 75% w/w.

According to another embodiment of this aspect, the chewing gum base is present in an amount from about 50% w/w to about 85% w/w, and the release-controlling polymer is present in an amount from about 10% w/w to about 50% w/w.

According to another embodiment of this aspect, the release-controlling polymer is polyethylene oxide.

According to another embodiment of this aspect, the chewing gum base and polyethylene oxide are present in a ratio of about 7:1 to about 1:1.

According to another embodiment of this aspect, the chewing gum base and polyethylene oxide are present in a ratio of about 5:1.

According to another embodiment of this aspect, the solid dosage form further comprises one or more aversive agents.

According to another embodiment of this aspect, the solid dosage may be in the form of caplets, pills, mini-tablets, tablets, or capsules; optionally caplets, pills, mini-tablets, or tablets can be filled into capsules.

According to another aspect, the present invention is a crush-resistant oral solid dosage form that consists essentially of:

a) a therapeutically effective amount of a drug prone to abuse; and

b) a chewing gum base comprising a chewable plastic polymer;

wherein the amount of said gum base is such that it causes the dosage form to deform plastically without breaking into powder when said dosage form is tampered for abuse.

According to another aspect, the present invention is a crush-resistant oral solid dosage form that consists essentially of:

a) a therapeutically effective amount of a drug prone to abuse;

b) a chewing gum base comprising a chewable plastic polymer; and

c) one or more aversive agents,

wherein the amount of said gum base is such that it causes the dosage form to deform plastically without breaking into powder when said dosage form is tampered for abuse.

A second aspect of the present invention provides a process for the preparation of a crush-resistant oral solid dosage form, the process comprising:
1) blending a drug prone to abuse, a chewing gum base, and optionally, one or more pharmaceutically acceptable excipients;
2) compressing the blend of step 1) to obtain caplets, pills, mini-tablets, or tablets;
3) optionally, filling the compressed blend of step 2) into capsules.

According to one embodiment of this aspect, the process further involves heating the compressed blend at a temperature of about 50° C. to about 80° C. for about 5 minutes to about 75 minutes.

A third aspect of the present invention provides a process for the preparation of a crush-resistant oral solid dosage form, the process comprising:
1) blending a drug prone to abuse, a chewing gum base, a release-controlling polymer, and optionally one or more pharmaceutically acceptable excipients;
2) compressing the blend of step 1) to obtain caplets, pills, mini-tablets, or tablets;
3) heating the compressed blend of step 2) in a coating pan at a temperature of about 50° C. to about 80° C. for about 5 minutes to about 75 minutes;
4) optionally, filling the heated compressed blend of step 3) into capsules.

According to one embodiment of this aspect, the process involves heating the compressed blend at a temperature of about 80° C. for about 10 minutes.

The terms “crush-resistant” or “crush-resistance,” as used herein, refer to the property of the dosage form that makes it less prone to being powdered or extracted to minimize the likelihood of abuse by snorting or extraction in a solvent for injection. The crush-resistant dosage form of the invention will deform plastically upon application of breaking force rather than disintegrating into powder form. It should be understand that in crushing the dosage form of the invention, there may be a small amount of powder formed, e.g., from the coating or other excipients present in the formulation, but at least a majority of the crushed dosage form will not be disintegrated into a powder form.

Crush-resistance in the dosage forms of the invention has been achieved by the use of chewing gum bases. The chewing gum bases comprise chewable plastic polymers to impart gummy and stretching properties to the dosage form. The commercially available chewing gum bases comprise plastic polymer (gum), sugar, anti-tackifiers, and plasticizers. In particular, the commercially available chewing gum bases containing chewable plastic polymers used in the present invention include PG Nutra PEPP 2T by Gum Base Co. and Health in Gum® PWD-03 by Cafosa.

The stretchable plastically deformed dosage form is unsuitable for snorting or inhalation. Further, the gummy plastic does not allow easy extraction of the drug in a solvent in sufficient quantity to be injected. Therefore, the potential for drug abuse is minimized through inhalation and injection route.

Release-controlling polymer(s) could be added to further improve the abuse-deterrent properties and to achieve the desired release profile.

The abuse-deterrent properties of the dosage form may be tested by the following techniques:

### Crushing Test

In the crushing test, the dosage form is subjected to crushing using a hammer, pestle-mortar, or an apparatus designed to measure the hardness of an oral dosage form. If the dosage form disintegrates into particles, then it may be possible to dissolve or suspend these particles or use the powder for snorting or sniffing and use them for abuse purposes.

However, if it is not possible to crush the dosage form in this test, then there will be no particles to use for such abuse purposes.

### Melting Test

In the melting test, the dosage form is subjected to heating, e.g., on a spoon or by exposure to microwave induced heating. If the dosage form melts to form a plastic or rubbery mass, the dosage form is not suitable for abuse purposes. However, if the dosage form liquefies so that it is possible to inject it without being too hot, then the dosage form may be prone to abuse.

### Extraction Test

In the extraction test, the dosage form is subjected to extraction in various solvents that are commonly available, e.g., water, ethanol, and those which have potentially relevant solvent characteristics (pH, polarity vs. aprotic, e.g., HCl). In the evaluation, the dosage form is crushed and contacted with a small amount of a solvent. After attempting to dissolve the dosage form, the volume is measured and the amount of the drug extracted is analysed and quantified. If a significant amount of drug is extracted such that it could then be injected by intravenous or subcutaneous routes to produce euphoric effects, then the dosage form may be prone to abuse.

Further, certain aversive agents may be added to the dosage form to cause disliking or aversion for subsequent use, if the dosage form is chewed to extract the drug for oral abuse. Usually, their amount ranges from about 0.1% w/w to about 1.0% w/w, in particular from about 0.2% w/w to about 0.5% w/w.

Unpalatable substances may include bittering agents or hot and pungent additives. Bittering agents may be selected from the group comprising quinine, sucrose octaacetate, quassin, brucine, quercetin, denatonium, and mixtures thereof. Pungent additives may be selected from capsaicinoids, e.g., capsaicin, dihydrocapsaicin, nordihydrocapsaicin, homocapsaicin, and homodihydrocapsaicin, to further deter oral drug abuse.

Irritants may be of natural or synthetic origin and include mustard and its derivatives, e.g., allyl isothiocyanate and p-hydroxybenzyl isothiocyanate; emetics, e.g., ipecac and chemotherapeutic agents; laxatives, e.g., aloe vera, bisacodyl, casanthranol, cascara sagrada, castor oil, dehydrocholic acid, phenolphthalein, senna, and sennosides.

Propylene glycol may be added additionally to the crush-resistant dosage form to provide a further abuse-deterrent effect. It has been found that when used via the injection route, propylene glycol produces immense pain and irritation. It also causes irritation of the respiratory system if inhaled, and leads to gastrointestinal distress upon accidental ingestion in large quantities. Hence, this may provide a further aversive effect in case the dosage form is successfully tampered with and the drug is extracted for injection. The amount of propylene glycol added may be from about 5% w/w to about 15% w/w, in particular about 10% w/w.

The dosage forms of the present invention include but are not limited to caplets, pills, capsules, mini-tablets, or tablets. Alternatively, the drug and excipients may be compressed to form mini-tablets or pills, which may then be filled into capsules. The dosage form of the present invention in the form of tablets can be made by wet granulation, dry granulation (e.g., slugging or roller compaction), direct compression, melt granulation, or hot-melt extrusion. The method of preparation and type of excipients may be selected based on the desired physical characteristics of the tablet formulation. Wet granulation may be carried out in the presence of aqueous or non-aqueous solvents. Non-aqueous solvents may be selected from ethanol, isopropyl alcohol, or suitable mixtures of ethanol:water and isopropyl alcohol:water.

Drugs prone to abuse or their pharmaceutically acceptable salts, derivatives, analogs or polymorphs may include psychoactive drugs and analgesics, including but not limited to opioids and drugs that can cause psychological and/or physical dependence on the drug. A drug for use in the present invention may be selected from alfentanil, amphetamines, bupropion, buprenorphine, butorphanol, carfentanyl, codeine, dezocine, diacetylmorphine, dihydrocodeine, dihydromorphine, diphenoxylate, diprenorphine, etorphine, fentanyl, hydrocodone, hydromorphone, levorphanol, lofentanil, meperidine, methadone, gabapentin, methylphenidate, morphine, oxycodone, oxymorphone, pentazocine, pethidine, propoxyphene, remifentanil, sufentanil, tilidine, tramadol, and salts, derivatives, analogs, homologues, and polymorphs thereof, or mixtures of any of the foregoing.

The dosage form of the present invention may in particular include one or more opioids, e.g., hydrocodone, morphine, oxymorphone, and oxycodone and/or salts thereof, as the therapeutically effective drug. Particularly, when processed into a suitable dosage form, the drug can be present in an amount ranging from about 0.5% w/w to about 30% w/w.

In addition, the dosage form may contain other pharmaceutically acceptable excipients to facilitate the manufacturing process. The other pharmaceutically acceptable excipients are selected from diluents, binders, lubricants, and mixtures thereof, and other excipients known to the person skilled in the art. Some of these excipients may result in a powder when the dosage form is crushed but the drug and chewing gum base generally cannot be crushed into a powder.

Binders may be selected from the group comprising microcrystalline cellulose, polyvinyl pyrrolidone, starch, maltrin, methyl cellulose, hydroxypropylmethyl cellulose, carboxymethyl cellulose, sucrose solution, dextrose solution, acacia, tragacanth, locust bean gum, pre-gelatinized starch, copovidone, shellac, zein, gelatin, polymethacrylates, synthetic resins, and mixtures thereof. Binders may be present in an amount from about 2% w/w to about 20% w/w.

Diluents may be selected from the group comprising lactose, microcrystalline cellulose, calcium hydrogen phosphate (dihydrate), calcium hydrogen phosphate (anhydrous), tribasic calcium phosphate, calcium carbonate, kaolin, magnesium carbonate, magnesium oxide, and mixtures thereof. Diluents may be present in amounts from about 5% w/w to about 40% w/w.

Lubricants may be selected from the group comprising stearic acid, polyethylene glycol, magnesium stearate, calcium stearate, zinc stearate, talc, sodium stearyl fumarate, and mixtures thereof. Lubricants may be present in an amount from about 0.1% w/w to about 2% w/w.

The dosage form of the invention may optionally be provided with a coating layer. The coating may be done with coating polymers, e.g., Eudragit®, ethyl cellulose, or HPMC. Organic or aqueous solvents may be used during the coating process. Solvents may be selected from the group comprising water, acetone, isopropyl alcohol, ethanol, methylene chloride, and mixtures thereof.

Plasticizers used during coating may be selected from the group comprising polyethylene glycols, dibutyl phthalate, diethyl phthalate, triethyl citrate, tributyl citrate, tributyrin, butyl phthalyl butyl glycolate, triacetin, castor oil, citric acid esters, and mixtures thereof. These plasticizers are present in an amount to facilitate the coating process and to obtain an even coating of film with enhanced physical stability. Generally, the coating material comprises from about 0.5% w/w to about 50% w/w of a plasticizer, particularly from about 10% w/w to about 20% w/w of the enteric polymer. Commercially available coatings may be employed, e.g., Opadry®. The tablet coating is about 2% w/w to about 5% w/w of the core tablet weight. In particular, about 3% w/w of the core tablet weight may be used for coating.

In addition, the coating material may also comprise inert solid particulates. In particular, talc and titanium dioxide are used as opacifiers. Lakes and dyes are also used to impart color to the coating. These include iron oxide (red or yellow), aluminum lakes, and natural colouring materials, e.g., anthocyanins, carotenoids.

The invention may be illustrated by the following non-limiting examples.

During the development stage, lactose monohydrate was used in the dosage forms (Example 1 to Example 11) in place of the drug prone to abuse, to evaluate the crush-resistance potential of the dosage form.

### EXAMPLES

### Examples 1-6

| | Amount Per Tablet (% w/w) | | | | | |
|---|---|---|---|---|---|---|
| Ingredients | 1 | 2 | 3 | 4 | 5 | 6 |
| Lactose | 10.00 | 10.00 | 10.00 | 10.00 | 10.00 | 10.00 |
| Chewing gum base | 20.00 | 44.00 | — | 69.00 | 69.00 | 44.00 |
| (PG Nutra PEPP 2T) | | | | | | |
| Chewing gum base | — | — | 44.00 | — | — | — |
| (Health in Gum ® | | | | | | |
| PWD 01) | | | | | | |
| Polyethylene oxide | 69.00 | 45.00 | 45.00 | 20.00 | — | — |
| (Polyox ® WSR-N80) | | | | | | |
| Hydroxypropylmethyl | — | — | — | — | 20.00 | — |
| cellulose | | | | | | |
| (Methocel ® K15 MCR) | | | | | | |
| Hydroxypropylmethyl | — | — | — | — | — | 45.00 |
| cellulose | | | | | | |
| (Methocel ® E10 MCR) | | | | | | |
| Carbopol ® 971P | — | — | — | — | — | — |
| Magnesium stearate | 1.00 | 1.00 | 1.00 | 1.00 | 1.00 | 1.00 |
| Isopropyl alcohol:water | — | — | — | — | — | — |
| Total (Core Tablet) | 100.00 | 100.00 | 100.00 | 100.00 | 100.00 | 100.00 |

* Opadry ® film coating was applied (3.00% w/w of the total weight of the core tablet) to all dosage forms of Examples 1-6

### Examples 7-11

| | Amount Per Tablet (% w/w) | | | | |
|---|---|---|---|---|---|
| Ingredients | 7 | 8 | 9 | 10 | 11 |
| Lactose | 10.00 | 10.00 | 10.00 | 10.00 | 10.00 |
| Chewing gum base | — | — | 69.00 | 20.00 | — |
| (PG Nutra PEPP ® 2T) | | | | | |
| Chewing gum base | 44.00 | 20.00 | — | — | 44.00 |
| (Health in Gum ® | | | | | |
| PWD 01) | | | | | |
| Polyethylene oxide | — | — | — | — | — |
| (Polyox ® WSR-N80) | | | | | |
| Hydroxypropylmethyl | — | — | — | — | — |
| cellulose | | | | | |
| (Methocel ® K15 MCR) | | | | | |
| Hydroxypropylmethyl | 45.00 | 69.00 | — | — | — |
| cellulose | | | | | |
| (Methocel ® E10 MCR) | | | | | |
| Carbopol ® 971P | — | — | 20.00 | 69.00 | 45.00 |
| Magnesium stearate | 1.00 | 1.00 | 1.00 | 1.00 | 1.00 |
| Isopropyl alcohol:water | — | — | — | q.s. | q.s. |
| Total Core Tablet | 100.00 | 100.00 | 100.00 | 100.00 | 100.00 |

* Opadry ® Film coating was applied (3.00% w/w of the total weight of the core tablet) to all dosage forms of Examples 7-11

### Manufacturing Process

### Examples 1-9

1) Lactose (substitute for drug prone to abuse) was mixed with chewing gum base (PG Nutra PEPP 2T/Health in Gum® PWD 01) and release-controlling polymer (Polyox® WSR-N80/Methocel® K15 MCR/Methocel® E10 MCR/Carbopol® 971P).
2) The blend of step 1) was lubricated using magnesium stearate and compressed using a suitable tablet press and tooling.
3) The compressed tablets of step 2) were film coated using an aqueous Opadry® coating dispersion in a tablet coating pan apparatus.

### Manufacturing Process

### Examples 10 and 11

1) Lactose was mixed with chewing gum base and Carbopol 971P.
2) The blend of step 1) was granulated with a mixture of isopropyl alcohol:water in a rapid mixer granulator.
3) The wet granules of step 2) were dried in a fluidized bed drier and milled to a suitable size.
4) The granules of step 3) were lubricated using magnesium stearate.
5) The lubricated blend of step 4) was compressed using a suitable tablet press and tooling.
6) The compressed tablets of step 5) were film coated using an aqueous Opadry® coating dispersion in a tablet coating pan apparatus.

Examples 1-11 were subjected to crushing test using a pestle-mortar in order to determine their ability to resist crushing upon tampering and misuse. The observations of the crushing test are provided in Table 1. These compositions may be extended to drugs prone to abuse by replacing lactose with the drug.

TABLE 1
Observations of Crushing Test for Examples 1 to 11

| | | | % Composition | | |
|---|---|---|---|---|---|
| | Chewing Gum | | Chewing | | Observation Upon |
| Ex. | Base Used | Polymer Used | Gum Base | Polymer | Crushing Test |
| 1 | PG Nutra PEPP | Polyethylene oxide | 20% | 69% | Hard mass which |
| | 2T | | | | cannot be broken |
| 2 | PG Nutra PEPP | | 44% | 45% | into a fine |
| | 2T | | | | powder |
| 3 | Health in Gum ® | | 44% | 45% | |
| | PWD-01 | | | | |
| 4 | PG Nutra PEPP | | 69% | 20% | |
| | 2T | | | | |
| 5 | PG Nutra PEPP | Hydroxypropylmethyl | 69% | 20% | Hard mass which |
| | 2T | cellulose | | | cannot be broken |
| | | (Methocel ® K15 MCR) | | | into a fine |
| 6 | PG Nutra PEPP | Hydroxypropylmethyl | 44% | 45% | powder |
| | 2T | cellulose | | | |
| 7 | Health in Gum ® | (Methocel ® E10 MCR) | 44% | 45% | |
| | PWD-01 | | | | |
| 8 | Health in Gum ® | Hydroxypropylmethyl | 20% | 69% | Gritty particles |
| | PWD-02 | cellulose | | | obtained |
| | | (Methocel ® E10 MCR) | | | |
| 9 | PG Nutra PEPP | Carbopol ® | 69% | 20% | Hard mass which |
| | 2T | | | | cannot be broken |
| | | | | | into a fine |
| | | | | | powder |
| 10 | PG Nutra PEPP | | 20% | 69% | Gritty particles |
| | 2T | | | | obtained |
| 11 | Health in Gum ® | | 44% | 45% | Hard mass which |
| | PWD-03 | | | | cannot be broken |
| | | | | | into a fine |
| | | | | | powder |

### Examples 12-15

| | Amount Per Tablet (% w/w) | | | |
|---|---|---|---|---|
| | Example | | | |
| Ingredients | 12 | 13 | 14 | 15 |
| Bupropion HCl | 12.50 | 12.50 | 12.50 | 12.50 |
| Lactose anhydrous | — | — | — | 30.00 |
| Avicel ® PH-102 | — | — | — | 31.00 |
| (Hydroxypropylmethyl cellulose) | 25.00 | — | — | 25.00 |
| Methocel ® K100M | | | | |
| Polyethylene oxide | — | 25.00 | 14.62 | — |
| (Polyox ® WSR-303) | | | | |
| Chewing gum base | 61.00 | 61.00 | 71.38 | — |
| (Health in Gum ® PWD-01) | | | | |
| Colloidal Silicon dioxide | 0.50 | 0.50 | 0.50 | 0.50 |
| (Aerosil ® 200) | | | | |
| Magnesium stearate | 1.00 | 1.00 | 1.00 | 1.00 |
| Total Core Tablet | 100.00 | 100.00 | 100.00 | 100.00 |

*Opadry ® Film coating (3.00% w/w of the total weight of the core tablet) to be applied to all dosage forms of Examples 12-14

### Manufacturing Process

### Examples 12-14

1) All the ingredients were accurately weighed.
2) Bupropion HCl, Methocel® K100 or Polyox® 303, and Health in Gum® PWD-01 were mixed in a suitable mixer and sifted through a sieve.
3) The material of step 2) was mixed in a blender for 5 minutes.
4) Aerosil® 200 and magnesium stearate were passed through a sieve.
5) The materials of step 3) and step 4) were mixed in a blender for 5 minutes.
6) The blend of step 5) was compressed into tablets.
7) The tablets of step 6) were heated at about 80° C. in a coating pan for 10 minutes to obtain pre-heated tablets.
8) The tablets of step 7) were film coated using an aqueous Opadry® coating dispersion in a tablet-coating pan apparatus.

### Manufacturing Process

### Example 15

1) All the ingredients were accurately weighed.
2) Bupropion HCl, lactose anhydrous, Methocel® K100M, and Avicel® PH-102 were mixed in a suitable mixer and the blend was sifted through a sieve.
3) The material of step 2) was mixed in a blender for 5 minutes.
4) Aerosil 200 and magnesium stearate were passed through a sieve.
5) The materials of step 3) and step 4) were mixed in a blender for 5 minutes.
6) The blend of step 5) was compressed into tablets.
7) The tablets of step 6) were film coated using aqueous Opadry® coating dispersion in a tablet-coating pan apparatus.

### Dissolution Study

The tablets of Examples 12-15 were subjected to dissolution. The amount of bupropion dissolved at time points 0.5, 1, 2, 3, 4, 6, and 8 hours is tabulated in Table 2.

TABLE 2
Dissolution Study data indicating %
drug release at various time points

| Time | | | | Example 15 |
|---|---|---|---|---|
| (Hours) | Example 12 | Example 13 | Example 14 | (Control) |
| 0.5 | 35 | 45 | 36 | 33 |
| 1 | 47 | 64 | 46 | 42 |
| 2 | 64 | 88 | 65 | 59 |
| 3 | 77 | 93 | 76 | 71 |
| 4 | 87 | 99 | 83 | 83 |
| 6 | 101 | — | 91 | 95 |
| 8 | — | — | 96 | 100 |

### Extraction Study

The tablets of Examples 12 and 15 were extracted in various solvents. The tablets were crushed with a mortar and pestle. The mass so obtained was then mixed with 30 mL of a solvent (water or ethanol). The contents were mixed for 30 minutes. The percentage amount of bupropion extracted at 15 minutes is tabulated in Table 3.

TABLE 3
Extraction Study data indicating the percentage of drug extracted

| Solvent | Example No. | % Drug Extracted |
|---|---|---|
| Water | Example 12 | 10.1 |
| | Example 15 | 36.9 |
| Ethanol | Example 12 | 45.44 |
| | Example 15 | 83.22 |

The tablets of Example 14 were extracted in water and ethanol according to the extraction method given above. The percentage amount of bupropion extracted at 15 minutes is tabulated in Table 4.

TABLE 4
Extraction Study data indicating the percentage of drug extracted

| Solvent | Example No. | % Drug Extracted |
|---|---|---|
| Water | Example 14 | 1.3 |
| Ethanol | Example 14 | 6.1 |

Based on the above experimental data, it was observed that the dosage form of the present invention comprising a chewing gum base possessed crush-resistance and were less prone to abuse.

We claim:

1. A crush-resistant oral solid dosage form, comprising:
a) a therapeutically effective amount of a drug prone to abuse; and
b) a chewing gum base comprising a chewable plastic polymer,
wherein the amount of said gum base is such that it causes the dosage form to deform plastically without breaking into powder when said dosage form is tampered with for abuse.

2. The solid dosage form according to claim 1, wherein the chewable plastic polymer present is selected from the group comprising polyisobutylene, butadiene styrene, polyvinyl acetate, terpene resins, ester gums, ethylene vinyl acetate, and mixtures thereof.

3. The solid dosage form according to claim 1, wherein the chewing gum base is present in an amount from about 20% w/w to about 85% w/w.

4. The solid dosage form according to claim 1, further comprising a release-controlling polymer.

5. The solid dosage form according to claim 4, wherein the release-controlling polymer is selected from the group comprising hydroxypropylmethyl cellulose, hydroxyethyl cellulose, polyethylene glycol, polyethylene oxide, hydroxypropyl cellulose, carboxymethyl cellulose, cellulose ethers, cellulose esters, polymethacrylic acid esters copolymers, aminoalkyl methacrylate copolymers, copolymers of polyvinyl acetate and polyvinyl pyrrolidone, polyvinyl alcohol, glyceryl behenate, carnauba wax, xanthan gum, starch, sodium alginate, tragacanth, poloxamers, carbomers, and mixtures thereof.

6. The solid dosage form according to claim 4, wherein the chewing gum base is present in an amount from about 20% w/w to about 85% w/w, and the release-controlling polymer is present in an amount from about 10% w/w to about 75% w/w.

7. The solid dosage form according to claim 6, wherein the chewing gum base is present in an amount from about 50% w/w to about 85% w/w, and the release-controlling polymer is present in an amount from about 10% w/w to about 50% w/w.

8. The solid dosage form according to claim 5, wherein the release-controlling polymer is polyethylene oxide.

9. The solid dosage form according to claim 8, wherein the chewing gum base and polyethylene oxide are present in a ratio of about 7:1 to about 1:1.

10. The solid dosage form according to claim 9, wherein the chewing gum base and polyethylene oxide are present in a ratio of about 5:1.

11. The solid dosage form according to claim 1, further comprising one or more aversive agents.

12. The solid dosage form according to claim 1, in the form of caplets, pills, mini-tablets, tablets, or capsules; optionally, caplets, pills, mini-tablets, or tablets can be filled into capsules.

13. The crush-resistant oral solid dosage form according to claim 1, wherein the dosage form consists essentially of (a) a therapeutically effective amount of a drug prone to abuse; and (b) a chewing gum base comprising a chewable plastic polymer in an amount such that it causes the dosage form to deform plastically without breaking into powder when said dosage form is tampered with.

14. A process for the preparation of a crush-resistant oral solid dosage form, the process comprising:
1) blending a drug prone to abuse, a chewing gum base, and optionally one or more pharmaceutically acceptable excipients;
2) compressing the blend of step 1) to obtain caplets, pills, mini-tablets, or tablets;
3) optionally, filling the compressed blend of step 2) into capsules.

15. The process according to claim 14, wherein the compressed blend is heated at a temperature of about 50° C. to about 80° C. for about 5 minutes to about 75 minutes.

16. A process for the preparation of a crush-resistant oral solid dosage form, the process comprising:
1) blending a drug prone to abuse, a chewing gum base, a release-controlling polymer, and optionally one or more pharmaceutically acceptable excipients;
2) compressing the blend of step 1) to obtain caplets, pills, mini-tablets, or tablets;
3) heating the compressed blend of step 2) in a coating pan at a temperature of about 50° C. to about 80° C. for about 5 minutes to about 75 minutes;
4) optionally, filling the heated compressed blend of step 3) into capsules.

17. The process according to claim 16, wherein the compressed blend is heated at a temperature of about 80° C. for about 10 minutes.
